(19) United States
(12) Patent Application Publication
(10) Pub. No.: US 2007/0059262 A1
(43) Pub. Date: Mar. 15, 2007

(54) Solid skin care composition comprising multiple layers

(76) Inventor: Toshiya Taniguchi, Kobe (JP)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/517,697

(22) Filed: Sep. 8, 2006

Related U.S. Application Data
(60) Provisional application No. 60/715,685, filed on Sep. 9, 2005.

(51) Int. Cl. A61K 8/89; A61K 8/36

(52) U.S. Cl. 424/63

(57) ABSTRACT

The present invention relates to a solid skin care composition comprising: (a) a first layer which is solid at 45° C. and which is selected from the group consisting of a water-in-oil emulsion, an oil-in-water emulsion and an oil dispersion; and (b) a second layer which is a pressed powder layer; wherein the first layer and the second layer are provided in the same package in a manner such that the first layer and the second layer can be simultaneously applied.

### CROSS REFERENCE TO RELATED APPLICATION

This application claims the benefit of U.S. Provisional Application No. 60/715,685 filed on Sep. 9, 2005.

### FIELD OF THE INVENTION

The present invention relates to a solid skin care composition comprising multiple layers. Specifically, the present invention relates to solid skin care compositions comprising multiple layers each made of different compositions providing unique characteristic benefits. The characteristic benefits would not be achieved to the extent when provided in separate phases, if the multiple layers were mixed together and provided as a single composition. The compositions of the present invention are particularly useful for cosmetic foundation products.

### BACKGROUND OF THE INVENTION

A foundation composition can be applied to the face and other parts of the body to even skin tone and texture and to hide pores, imperfections, fine lines and the like. A foundation composition is also applied to moisturize the skin, to balance the oil level of the skin, and to provide protection against the adverse effects of sunlight, wind, and other environmental factors.

Foundation compositions are generally available in the form of liquid or cream suspensions, emulsions, gels, pressed powders or anhydrous oil and wax compositions. Emulsion-type foundations in the form of liquid are suitable in that they provide moisturizing effects by the water and water-soluble skin treatment agents incorporated. These liquid form foundations, however, are less convenient to use and carry for the consumer. On the other hand, solid foundations packaged in compacts are suitable for use by the consumer, however, are typically less efficient than liquid form foundations in terms of moisturizing the skin and coverage of the skin.

Foundation compositions in the form of solid, yet emulsion have been suggested. Such solid emulsion foundations aim to address the drawbacks of conventional liquid form foundations and solid foundations. These foundations can be filled in a wide variety of packaging, including compacts, and is increasing popularity among consumers. References which disclose such foundation compositions include Japanese patent publications A-2-8851 1, A-3-261707, A-7-267819, A-1 1-209243, U.S. Pat. No. 5,362,482, and PCT publication WO 01/91704.

Recently, consumers have become to seek various performances and benefits in foundation products, such as radiant look, natural look, spreadability; fit to the skin, blending into the skin, coverage, wear, long lasting, oil shine control, UV protection, and specific treatment provided by skin active agents. Further, different consumer segments may seek different types of performance, such as moisturizing feel against light feel, and natural look against lusterous finish. To achieve these benefits, foundation formulations must accommodate various components which, depending on their physical and chemical properties, may be difficult to formulate into a single product. For example, oil shine control is a highly desirable function for a foundation product. However, incorporation of oil absorbing powders at a high level will render the formulation to have a very heavy application feel with poor spreadability. Incorporation at a high level may also make the emulsion unstable.

On the other hand, cosmetic compositions comprising multiple layers or phases are known in the prior art. These products are usually provided in the phase type of cream, gel, or paste and are usually focusing on the distinctness of the color of each layer. For example, U.S. Pat. No. 4,980,155 to Revlon, Inc. discloses a two phase cosmetic composition comprising a color phase composition and a gel phase composition. WO2004/105708 to Gamma Croma S.P.A. discloses a multicolor cosmetic product with solid consistence that comprises two or more cosmetic products of different colors. JP Patent Application Publication No. 1999-269025 to Noevir Co., Ltd. discloses a double-layered stick-shaped cosmetic product comprising an oil-based stick-shaped composition and a water-based stick-shaped composition. JP Patent Application Publication No. 2002-97112 discloses a solid cosmetic composition having mutually different colors and the manufacturing process for the same. None of them disclose a multi-layered skin care composition which is in the form of solid water-in-oil emulsions in ambient temperature.

Based on the foregoing, there is a need for a solid skin care composition which provides more than one benefit rendered by components which are difficult to formulate into a single composition. Specifically for cosmetic foundation products, there is a need for a solid composition which provides good spreadability, oil shine control and wear benefits in one product.

None of the existing art provides all of the advantages and benefits of the present invention.

### SUMMARY OF THE INVENTION

The present invention is directed to a solid skin care composition comprising: a) a first layer which is solid at 45° C. and which is selected from the group consisting of a water-in-oil emulsion, an oil-in-water emulsion and an oil dispersion; and b) a second layer which is a pressed powder; wherein at least one of the first layer and the second layer comprises a benefit agent and wherein the first layer and the second layer are provided in the same package in a manner such that the first layer and the second layer can be simultaneously applied.

By providing multiple layers of compositions in a manner such that they can be simultaneously applied, the overall composition provides benefits characteristic of each layer, which benefit(s) would otherwise be compromised or deteriorate other performance, if they were combined into one composition.

The present invention is suitable for any skin care composition in solid form, such as cosmetic foundation, blusher, sunscreen, eyeshadow and others. One particularly preferred embodiment for the present invention is a cosmetic foundation made of multiple layers that are visibly distinct.

These and other features, aspects, and advantages of the present invention will become evident to those skilled in the art from a reading of the present disclosure with the appended claims.

### DETAILED DESCRIPTION OF THE INVENTION

While the specification concludes with claims particularly pointing out and distinctly claiming the invention, it is believed that the present invention will be better understood from the following description.

All percentages, parts and ratios as used herein are by weight of the composition of each layer of the present invention, unless otherwise specified. All such weights as they pertain to listed ingredients are based on the active level and, therefore do not include carriers or by-products that may be included in commercially available materials.

All ingredients such as actives and other ingredients useful herein may be categorized or described by their cosmetic and/or therapeutic benefit or their postulated mode of action. However, it is to be understood that the active and other ingredients useful herein can, in some instances, provide more than one cosmetic and/or therapeutic benefit or operate via more than one mode of action. Therefore, classifications herein are made for the sake of convenience and are not intended to limit an ingredient to the particularly stated application or applications listed.

First Layer and Second Layer

The composition of the present invention comprises multiple layers, namely at least a pressed powder layer and a layer selected from the group consisting of a water-in-oil emulsion, an oil-in-water emulsion and oil dispersion. For the convenience of description, the layer selected from water-in-oil emulsion, oil-in-water emulsion and oil dispersion is named as the first layer, and the pressed powder layer is named as the second layer. By providing multiple layers of compositions in a manner such that they can be simultaneously applied, the overall composition provides benefits characteristic of each layer, which benefit(s) would otherwise be compromised or deteriorate other performance, if they were combined into one composition. While any number of layers can be included in the overall composition, an overall composition having two layers is focused in the discussion herein.

The first layer and second layer are of different composition, and are designed to provide different benefits based on at least one benefit agent included in either of the layers. The first layer and second layer may comprise different benefit agents, different combination of benefit agents, or different concentrations of the same benefit agent. In the context of the present invention, a “benefit agent” is a component which provides a particular skin care benefit characteristic of the usage of the skin care product. Herein, skin care benefit may include benefits related to appearance or make-up of the skin. Typically, a certain benefit agent included in one layer is less compatible with a certain component included in the other layer, or a certain benefit agent in one layer deteriorates performance of the overall composition when the first and second layers are combined into one composition.

For example, oil shine control -and good spreadability are two favorable characteristics for a cosmetic foundation composition, however, are difficult to achieve in one product. Oil shine control can be provided by a composition comprising a high level of powder, such as by a pressed powder of the second layer. On the other hand, good spreadability can be provided by an oil-in-water, water-in-oil, or oil dispersion type composition of the first layer. By combining the first layer and the second layer in a manner such that they can be simultaneously applied, two characteristics, which would otherwise be difficult to achieve, can be provided in one product.

In another example, incorporation of skin care agents in a cosmetic foundation composition is preferred. However, it is difficult to incorporate skin care agents in a meaningful amount in a pressed powder. By incorporating the skin care agent in the first layer and combining with the second layer, a product containing sufficient amount of skin care agent, while having the favorable characteristics of a pressed powder can be obtained in one product.

The first layer and second layer of the present invention are solid at room temperature, thus do not, or only slightly dissolve or mingle with each other during storage, and after each use. The first layer and the second layer are provided in a manner that allows the user to simultaneously apply both layers to the skin. A suitable way is to provide both layers in the same primary package, for example a pan, jar, or stick applicator. The primary package may accompany a suitable applicator, such as a sponge or brush. Preferably, the first and second layers are formulated such that they exhibit a similar rheology profile when receiving pressure/heat from the finger or applicator upon use.

The first layer and the second layer can be provided in any ratio as necessary for providing the target benefit(s). Preferably, the first layer and the second layer are provided in a weight ratio of from about 1:99 to about 99:1, more preferably from about 1:9 to about 9:1. The first layer and the second layer are preferably visibly distinct, so that the different benefits/characteristics of the layers are communicated to the user. A colorant may be suitably included in at least one of the first or second layers for making the layers visibly distinct.

Phase Type and Formulation of First Layer and Second Layer

In the present invention, the first layer composition has a phase type selected from the group consisting of water-in-oil emulsion, oil-in-water emulsion and oil dispersion and the second layer composition has a phase type of pressed powder. Water-in-oil emulsion, oil-in-water emulsion and oil dispersion are useful for providing good application feel to the skin, while also being able to encompass oil soluble or water soluble skin care agents, and further leaving a fresh and cool feeling after the water and/or volatile oils is evaporated. Pressed powder is useful for incorporating high level of powders which may provide, for example, oily shine control benefit and soft focus benefit. In one highly preferred embodiment, the present composition is a cosmetic foundation.

The pressed powder compositions of the present invention preferably comprise the following components:

(a) from about 60% to about 98%, more preferably from about 75% to about 95% of a pigment powder; and

(b) from about 2% to about 40%, more preferably from about 5% to about 25% of a non-volatile oil

(c) optional volatile silicone, when present, from about 0.1% to about 10%, more preferably from about 1% to about 5%.

(d) optional lipophilic surfactant, when present, from about 0.1% to about 7%, more preferably from about 0.3% to 5%.

When the first layer is a water-in-oil emulsion, the first layer composition preferably comprises the following components:

(a) from about 10% to about 50%, more preferably from about 15% to about 35% of a volatile silicone oil;

(b) from about 1% to about 10%, more preferably from about 2% to about 5% of a solid wax;

(c) from about 5% to about 45%, more preferably from about 15% to about 30% of a pigment powder;

(d) from about 0.5% to about 20%, more preferably from about 1% to about 15% of a non-volatile oil;

(e) from about 0.5% to about 5%, more preferably from about 1% to about 4% of a lipophilic surfactant; and

(f) an amount of water, such that the total level of the volatile silicone oil and water is more than about 40%, preferably from about 10% to about 35% of water.

When the first layer is an oil-in-water emulsion, the first layer composition preferably comprises the following components:

(a) from about 20% to about 60%, more preferably from about 30% to about 50% of water;

(b) from about 0.1% to about 4%, more preferably from about 0.3% to about 2% of a hydrophilic surfactant;

(c) from about 5% to about 40%, more preferably from about 10% to about 30% of a pigment powder;

(d) from about 1% to about 20%, more preferably from about 5% to about 15% of a non-volatile oil;

(e) from about 1% to about 15%, more preferably from about 2% to about 10% of a fatty compounds or fatty acid salts; and

(f) from about 1% to about 30%, more preferably from about 5% to about 20% of a volatile silicone oil.

When the first layer is oil dispersion, the first layer composition preferably comprises the following components:

(a) from about 10% to about 80%, more preferably from about 20% to about 70% of a volatile silicone oil;

(b) from about 1% to about 40%, more preferably from about 5% to about 25% of a non-volatile oil;

(c) from about 1% to about 10%, more preferably from about 2% to about 7% of a solid wax; and

(d) optional pigment powder, when present, from about 1% to about 70%, more preferably from about 5% to about 50% of a pigment powder.

At least one of the first layer and the second layer further comprises at least one benefit agent selected from the group consisting of radiant powder, soft focus powder, soft focus silicone elastomer, film forming polymer, oil absorbing powder, sebum solidifying powder, skin active agent and mixtures thereof. Preferably, when incorporated, the first layer comprises one or more benefit agents selected from the group consisting of radiant powder, soft focus powder, oil absorbing powder, sebum solidifying powder and mixtures thereof. Preferably, when incorporated, the second layer comprises one or more benefit agents selected from the group consisting of soft focus silicone elastomer, film forming polymer, skin active agent and mixtures thereof. Water-in-oil emulsion, oil-in-water emulsion and oil dispersion compositions are formulated to have a viscosity of from about 100 mPas to about 10,000 mPas, preferably from about 300 mPas to about 3,000 mPas when brought to a temperature of between about 55° C. and about 90° C.

Benefit Agent

The composition of the present invention comprises a benefit agent which provides a particular skin benefit characteristic of the usage of the skin care product. Herein, skin care benefit may include benefits related to appearance or make-up of the skin.

In a cosmetic composition embodiment, including but not limited to cosmetic foundation, blusher, sunscreen, eyeshadow, the benefit agent is selected from the group consisting of radiant powder, oil absorbing powder, film forming polymer, soft focus powder, soft focus silicone elastomer, sebum solidifying powder, skin active agent and mixtures thereof.

Radiant Powder

Radiant powder is a pigment that is particularly effective in providing radiant look to the skin, by having a gloss level of more than 7.0. Gloss level is a parameter which can be measured by a known method using the opacity charts available from THE LENETA COMPANY, Drawdown bar (0.003 μm and 0.006 μm), solvent (KP-545 available from Shin-Etsu Chemical Co., Ltd.), Gloss Checker IG-320 available from HORIBA.

The radiant powders useful herein include pearl pigments, mica, synthetic mica, boron nitride and specified particle talc having an average particle size of about 20 μm and a gloss level of about 7.2 (0.003 μm on white back), about 33.0 (0.006 μm on white back), about 8.5 (0.003 μm on black back) and about 10.3 (0.006 μm on black back). Commercially available specified particle talc can be obtained from Miyoshi Kasei Inc. under the trade name of SI-TALC CT-20. Specified particle talc has a higher gloss level and a lower transparency level than normal particle talc. Specifically, the gloss level of specified particle talc is about 130% to 200% vs. normal particle talc and the transparency level of specified particle talc is about 10% to 100% vs. normal particle talc. Transparency level can be measured by a known method using the opacity charts available from THE LENETA COMPANY, Drawdown bar (0.003 um and 0.006 um), solvent (KP-545 available from Shin-Etsu Chemical Co., Ltd.), Spectraflash available from Datacolor.

In a single layer formulation, because other powders, such as coverage titanium dioxide contained in the formulation may overwhelm the radiant powders effect, to achieve the radiant look effect, a typical level of radiant powders is as high as 5%. As used in the present application, coverage titanium dioxides are those having a particle size of from about 200 nm to about 500 nm. If the particle size is out of this range, the titanium dioxide may not provide enough coverage as a cosmetic material. In the present invention, by formulating the radiant powders mainly in one layer and coverage titanium dioxide in the other layer, and providing the two layers in a manner such that they can be simultaneously applied on the skin, the skin care product of the present invention can provide satisfied radiant appearance effect with lower level of radiant powders. As a result, there is provided more flexibility in product formulation. Compared to a single layer product, a multiple layer product comprising lower level of radiant powder having a better spreadbility and light feel on the skin is obtained. In a preferred example, the content level of radiant powders in one layer is from about 5% to about 25%, more preferably from about 10% to about 20% by weight of the composition of that layer. When calculated based on the total weight of the first layer and the second layer, the preferred content level of radiant powders is from about 0.5% to about 4%, more preferably from about 1% to about 3%.

Soft Focus Agent (1) Soft Focus Powder

Soft focus powder is a pigment that is particularly effective in providing a soft focus effect to the composition, namely natural finish yet having good coverage for minimizing the appearance of skin troubles, when incorporated in a defined amount. Specifically, the soft focus powder herein must meet two parameter criteria to provide such an effect. First, both the Total Luminous Transmittance (Tt) and Diffuse Luminous Transmittance (Td) of the pigment are relatively high. The soft focus powder has a Total Luminous Transmittance (Tt) of from about 40 to about 94 and a Diffuse Luminous Transmittance (Td) of from about 28 to about 38. Without being bound by theory, it is believed that, by having such high Tt and Td values, the soft focus powders exhibit a high transparency, thereby providing an overall natural finish. Second, the soft focus powder has a relatively high Haze value {(Td/Tt)×100} of from about 32 to about 95. Without being bound by theory, it is believed that, by having such high Haze value, the contrast between lighted area of the skin and shaded area of the skin (such as pores and wrinkles) is minimized for reducing the appearance of the trouble areas.

Total Luminous Transmittance (Tt), Diffuse Luminous Transmittance (Td), and Haze value {(Td/Tt) x 100} can be measured and calculated by the artisan by reference to ASTM D 1003-00 “Standard Test Method for Haze and Luminous Transmittance of Transparent Plastics”. Although the pigments herein are not plastics, the same principles of this specific standard test can be applied.

The soft focus powder useful herein includes polymethyl/methacrylate (PMMA), silica, hybrid pigments such as alumina treated mica, titanium dioxide treated talc, titanium dioxide treated mica, vinyl dimethicone/methicone silsesquioxane crosspolymer, alumina, barium sulfate and synthetic mica. Commercially available soft focus powder useful herein includes alumina treated mica under the trade name of SA Excel Mica JP2 available from Miyoshi Kasei, which has a Total Luminous Transmittance (Tt) of about 87, Diffuse Luminous Transmittance (Td) of about 28, and Haze value {(Td/Tt)×100} of about 32.

Similar to the radiant powders, when formulated with coverage titanium dioxide in a single layer, the content level of a soft focus powder shall be as high as 5% to achieve noticeable natural look effect. However, in the present invention, by formulating a soft focus powders mainly in one layer and coverage titanium dioxide in the other layer, and providing the two layers in a manner such that they can be simultaneously applied on the skin, the skin care product of the present invention can provide satisfied natural look effect with lower level of soft focus powder. As a result, the cost of the product can be controlled while also providing more flexibility in product formulation. In a preferred example, the content level of soft focus powders in one layer is from about 2% to about 25%, more preferably from about 5% to about 20% based on the composition of that layer. When calculated based on the total weight of the first layer and the second layer, the preferred content level of soft focus powders is from about 0.5% to about 4%, more preferably from about 1% to about 3%.

Soft Focus Agent (2) Silicone Elastomer

Soft focus silicone elastomer is crosslinked siloxane elastomer that is particularly effective in providing soft focus effect to the skin. In other words, when incorporated in a cosmetic product a defined amount of silicone elastomer, the silicone elastomer can provide natural finish yet having good coverage for minimizing the appearance of skin troubles. Specifically, silicone elastomer has lower matte level compared with other silicone oils. Matte level is a parameter reflecting soft focus effect, i.e. natural finish of a cosmetic material. The lower is the matte level, the better natural finish the material can provide. Matte level of the silicone elastomer used herein is less than 40. Matte level can be measured by the PG-1M gloss meter (Incidence angle / Reflection angle: 60/60°) made by Nihon Denshoku Kogyo. Commercially available silicone elastomer useful in the present application includes a silicone elastomer having the trade name KSG-16 available from Shinetsu, which has a matte level of about 37.

Silicone elastomers suitable for use herein can be emulsifying or non-emulsifying crosslinked siloxane elastomers or mixtures thereof. The term “non-emulsifying” as used herein, defines crosslinked organopolysiloxane elastomers from which polyoxyalkylene units are absent. The term “emulsifying” as used herein, means crosslinked organopolysiloxane elastomers having at least one polyoxyalkylene (e.g., polyoxyethylene or polyoxypropylene) unit. Non-emulsifying elastomers useful in the present invention are formed via crosslinking organohydroenpolysiloxanes with an alpha, omega-diene. Emulsifying elastomers herein include polyoxyalkylene modified elastomers formed via crosslinking from organohydrogenpolysiloxanes with polyoxyalkylene dienes or organohydrogenpolysiloxanes containing at least one polyether group crosslinked with an alpha, omega-diene. Emulsifying crosslinked organopolysiloxane elastomer can notably be chosen from the crosslinked polymers described in U.S. Pat. Nos. 5,412,004, 5,837,793, and 5,811,487. In addition, an emulsifying elastomer comprised of dimethicone copolyol crosspolymer (and dimethicone) is available from Shin Etsu under the tradename KSG-21.

Non-emulsifying elastomers are dimethicone/vinyl dimethicone crosspolymers. Such dimethicone/vinyl dimethicone crosspolymers are supplied by a variety of suppliers including Dow Coming (DC 9040 and DC 9041), General Electric (SFE 839), Shin Etsu (KSG-15, 16, 18 [dimethicone/phenyl vinyl dimethicone crosspolymer]), and Grant Industries (GRANSIL™ line of elastomers). Cross-linked organopolysiloxane elastomers useful in the present invention and processes for making them are further described in U.S. Pat. Nos. 4,970,252, 5,760,116, and 5,654,362. Additional crosslinked organopolysiloxane elastomers useful in the present invention are disclosed in Japanese Patent Application JP 61-18708, assigned to Pola Kasei Kogyo K K. Commercially available elastomers preferred for use herein are Dow Coming's 9040 silicone elastomer blend, Shin Etsu's KSG-21, and mixtures thereof.

Similar to the radiant powders, when formulated with coverage titanium dioxide powders in a single layer, the content level of a silicone elastomer shall be as high as 10% to achieve noticeable natural look effect. However, in the present invention, by formulating a silicone elastomer mainly in one layer and coverage titanium dioxide in the other layer, and providing the two layers in a manner such that they can be simultaneously applied on the skin, the skin care product of the present invention can provide satisfied natural look effect with lower level of silicone elastomer. As a result, the cost of the product can be controlled while also providing more flexibility in product formulation. In a preferred example, the content level of silicone elastomer in one layer is from about 1% to about 35%, preferably from about 2% to about 25%. When calculated based on the total weight of the first layer and the second layer, the preferred content level of silicone elastomer is from about 0.5% to about 8%, more preferably from about 1% to about 7%.

Oil Absorbing Powder

Oil absorbing powder is a pigment that is particularly effective in absorbing oil, and thereby can be included in the present composition for absorbing excessive sebum from the skin. Specifically, the oil absorbing powder herein has an oil absorbency of at least about 100 ml/100 g, preferably at least about 200 ml/100 g. Oil absorbency is a unit well known to the artisan, and which can be measured via: JIS K5101 No.21 “Test Method for Oil Absorbency Level”.

Oil absorbing powder useful herein includes spherical silica, and methyl methacrylate copolymer. Commercially available oil absorbing pigments useful herein include spherical silica with tradename SI-SILDEX H-52 available from Miyoshi Kasei, Inc. having an oil absorbency of more than 200 ml/100 g, vinyl dimethicone/methicone silsesquioxane crosspolymer with tradename KSP-100 and KSP-101 available from ShinEtsu Chemical having an oil absorbency of more than 200 ml/100 g, hardened polyorgano siloxane elastomers with tradename TREFIL E-506C available from Dow Coming having an oil absorbency of more than 100 ml/100 g, and methyl methacrylate copolymer with tradename SA-GMP-0820 available from GANZ Chemical and surface treated by Miyoshi Kasei, Inc. having an oil absorbency of more than 100 ml/100 g. Typically, inclusion of oil absorbing powders for oil shine control may provide a composition with unfavorable spreadability performance. However, in the present invention, by including oil absorbing powders mainly in one layer, the unfavorable spreadability performance can be improved. In a preferred embodiment, one layer of the present invention comprises from about 1% to about 20%, more preferably from about 2% to about 10% of an oil absorbing powder.

Film Forming Polymer

Film forming polymer is useful for imparting wear and/or transfer resistant properties to a cosmetic product. Preferred polymers form a non-tacky film which is removable with water used with cleansers such as soap.

Examples of suitable film forming polymeric materials include:
a) sulfopolyester resins, such as AQ sulfopolyester resins, such as AQ29D, AQ35S, AQ38D, AQ38S, AQ48S, and AQ55S (available from Eastman Chemicals);
b) polyvinylacetate/polyvinyl alcohol polymers, such as Vinex resins available from Air Products, including Vinex 2034, Vinex 2144, and Vinex 2019;
c) acrylic resins, including water dispersible acrylic resins available from National Starch under the trade name “Dermacryl”, including Dermacryl LT;
d) polyvinylpyrrolidones (PVP), including Luviskol K17, K30 and K90 (available from BASF), water soluble copolymers of PVP, including PVP/JVA S-630 and W-735 and PVP/dimethylaminoethylmethacrylate Copolymers such as Copolymer 845 and Copolymer 937 available from ISP, as well as other PVP polymers disclosed by E. S. Barabas in the Encyclopedia of Polymer Science and Engineering, 2 Ed. Vol. 17 pp. 198-257;
e) high molecular weight silicones such as dimethicone and organic-substituted dimethicones, especially those with viscosities of greater than about 50,000 mPas;
f) high molecular weight hydrocarbon polymers with viscosities of greater than about 50,000 mPas;
g) organosiloxanes, including organosiloxane resins, fluid diorganopolysiloxane polymers and silicone ester waxes.

Examples of these polymers and cosmetic compositions containing them are found in PCT publication Nos. WO96/33689, published 10/31/96; WO97/17058, published 5/15/97; and U.S. Pat. No. 5,505,937 issued to Castrogiovanni et al. Apr. 9, 1996, all incorporated herein by reference. Additional film forming polymers suitable for use herein include the water-insoluble polymer materials in aqueous emulsion and water soluble film forming polymers described in PCT publication No. WO98/18431, published May 7, 1998, incorporated herein by reference. Examples of high molecular weight hydrocarbon polymers with viscosities of greater than about 50,000 mPas include polybutene, polybutene terephthalate, polydecene, polycyclopentadiene, and similar linear and branched high molecular weight hydrocarbons.

Preferred film forming polymers include organosiloxane resins comprising combinations of R₃SiO_{1/2} “M” units, R₂SiO “D” units, RSiO_{3/2} “T” units, SiO₂ “Q” units in ratios to each other that satisfy the relationship RₙSiO_{(4-n)/2} where n is a value between 1.0 and 1.50 and R is a methyl group. Note that a small amount, up to 5%, of silanol or alkoxy functionality may also be present in the resin structure as a result of processing. The organosiloxane resins must be solid at about 25° C. and have a molecular weight range of from about 1,000 to about 10,000 grams/mole. The resin is soluble in organic solvents such as toluene, xylene, isoparaffins, and cyclosiloxanes or the volatile carrier, indicating that the resin is not sufficiently crosslinked such that the resin is insoluble in the volatile carrier. Particularly preferred are resins comprising repeating monofunctional or R₃SiO_{1/2} “M” units and the quadrofunctional or SiO₂ “Q” units, otherwise known as “MQ” resins as disclosed in U.S. Pat. No. 5,330,747, Krzysik, issued Jul. 19, 1994, incorporated herein by reference. In the present invention the ratio of the “M” to “Q” functional units is preferably about 0.7 and the value of n is 1.2. Organosiloxane resins such as these are commercially available such as Wacker 803 and 804 available from Wacker Silicones Corporation of Adrian Michigan, KP545 from Shin-Etsu Chemical and G. E. 1170-002 from the General Electric Company. In the present invention, by having film forming polymer mainly in one layer, the film forming polymer will exist in a higher concentration at a localized area, and thereby forming a film of higher film intensity when applied to the skin, compared to the remainder of the composition. Such concentrated area of high film intensity provides improved adhesion of the entire composition to the skin. Namely, by providing the film forming polymer mainly in one layer, the amount of film forming polymer included in the entire composition can be reduced, or if the same amount of film forming polymer is formulated in the second layer, an entire composition having improved adhesion is obtained. In a preferred embodiment, one layer of the present invention comprises from about 0.5% to about 20%, more preferably from about 1% to about 8% of a film forming polymer.

Sebum Solidifying Powder

Sebum solidifying powder useful herein includes those comprising a base substance which is coated with low crystalline zinc oxide, amorphous zinc oxide, or mixtures thereof, wherein the zinc oxide is from about 15% to about 25% by weight of the sebum solidifying powder. The base substance may be any organic or inorganic substances that are useful for cosmetic use, including those listed below under “Pigment Powder Component”. The sebum solidifying powder herein can be suitably made according to the methods disclosed in US 2002/0031534 A1, herein incorporated by reference. The sebum solidifying powders may be surface treated. The sebum solidifying powders useful herein have the ability to solidify sebum, i.e., are effective in adsorbing free fatty acid, diglyceride, and triglyceride, and solidifying them by forming zinc salts thereof, such that a film is formed within about 30 minutes. Moreover, the originally glossy sebum changes appearance into a matte film. Such capability can be distinguished from other oil absorbing powders, which are not selective in the type of oil to be absorbed, and do not form a film after absorbing oil, thus may leave glossy gels and pastes after absorbing the sebum. Change in appearance provides a noticeable signal to the user that sebum has been controlled. Sebum solidifying effect may be conveniently measured by mixing a certain amount of powder with a certain amount of artificial sebum, mixing for a certain period of time, and allowing standing until solidified or showing matte appearance. The time taken for the mixture to solidify or to change appearance is recorded. The shorter the time taken to solidify or change appearance, the higher the solidifying effect is of the powder.

Commercially available sebum solidifying powder useful herein include mica coated with hydroxyapatite, 20% zinc oxide with tradename PLV-20, and the same powder surface treated with Methicone with tradename SI-PLV-20, both available from Miyoshi Kasei, Inc. Typically, inclusion of sebum solidifying powders for oil shine control may provide a composition with unfavorable spreadability performance. However, in the present invention, by including sebum solidifying powders mainly in one layer, the unfavorable spreadability performance can be improved. In a preferred example, the content level of sebum solidifying powder in one layer is from about 1% to about 35%, preferably from about 1% to about 25%.

Skin Active Agent

Skin active agent means an active ingredient which provides a cosmetic and/or therapeutic effect to the area of application on the skin. Compositions of the present invention may comprise a safe and effective amount of a skin active agent. The skin active agents useful herein include skin lightening agents, anti-acne agents, emollients, non-steroidal anti-inflammatory agents, topical anaesthetics, artificial tanning agents, antiseptics, anti-microbial and anti-fungal actives, skin soothing agents, sun screening agents, skin barrier repair agents, anti-wrinkle agents, anti-skin atrophy actives, lipids, sebum inhibitors, sebum inhibitors, skin sensates, protease inhibitors, skin tightening agents, anti-itch agents, hair growth inhibitors, desquamation enzyme enhancers, anti-glycation agents, and mixtures thereof. When included, one layer of the present composition comprises from about 0.001% to about 30%, preferably from about 0.001% to about 10% of at least one skin active agent.

The type and amount of skin active agents are selected so that the inclusion of a specific agent does not affect the stability of the composition. For example, hydrophilic agents may be incorporated in an amount soluble in the aqueous phase, while lipophilic agents may be incorporated in an amount soluble in the oil phase.

Skin lightening agents useful herein refer to active ingredients that improve hyperpigmentation as compared to pre-treatment. Useful skin lightening agents herein include ascorbic acid compounds, vitamin B₃ compounds, azelaic acid, butyl hydroxyanisole, gallic acid and its derivatives, glycyrrhizinic acid, hydroquinone, kojic acid, arbutin, mulberry extract, and mixtures thereof. Use of combinations of skin lightening agents is believed to be advantageous in that they may provide skin lightening benefit through different mechanisms.

Ascorbic acid compounds useful herein include ascorbic acid per se in the L-form, ascorbic acid salt, and derivatives thereof. Ascorbic acid salts useful herein include, sodium, potassium, lithium, calcium, magnesium, barium, ammonium and protamine salts. Ascorbic acid derivatives useful herein include, for example, esters of ascorbic acid, and ester salts of ascorbic acid. Particularly preferred ascorbic acid compounds include 2-o-D-glucopyranosyl-L-ascorbic acid, which is an ester of ascorbic acid and glucose and usually referred to as L-ascorbic acid 2-glucoside or ascorbyl glucoside, and its metal salts, and L-ascorbic acid phosphate ester salts such as sodium ascorbyl phosphate, potassium ascorbyl phosphate, magnesium ascorbyl phosphate, and calcium ascorbyl phosphate. Commercially available ascorbic compounds include magnesium ascorbyl phosphate available from Showa Denko, 2-o-D-glucopyranosyl-L-ascorbic acid available from Hayashibara and sodium L-ascorbyl phosphate with tradename STAY C available from Roche.

Vitamin B₃ compounds useful herein include, for example, those having the formula: 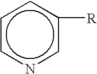
wherein R is —CONH₂ (e.g., niacinamide) or —CH₂OH (e.g., nicotinyl alcohol); derivatives thereof; and salts thereof. Exemplary derivatives of the foregoing vitamin B₃ compounds include nicotinic acid esters, including non-vasodilating esters of nicotinic acid, nicotinyl amino acids, panthenol, nicotinyl alcohol esters of carboxylic acids, nicotinic acid N-oxide and niacinamide N-oxide. Preferred vitamin B₃ compounds are niacinamide and tocopherol nicotinate, and more preferred is niacinamide. In a preferred embodiment, the vitamin B₃ compound contains a limited amount of the salt form and is more preferably substantially free of salts of a vitamin B₃ compound. Preferably the vitamin B₃ compound contains less than about 50% of such salt, and is more preferably essentially free of the salt form. Commercially available vitamin B₃ compounds that are highly useful herein include niacinamide USP available from Reilly.

Other skin active agents useful herein include N-acetyl glutathione (for example, N-acetyl glutathione available from Technical Sourcing International).

Other hydrophobic skin lightening agents useful herein include ascorbic acid derivatives such as ascorbyl tetraisopalmitate (for example, VC-IP available from Nikko Chemical), ascorbyl palmitate (for example available from Roche Vitamins), ascorbyl dipalmitate (for example, NIKKOL CP available from Nikko Chemical); undecylenoyl phenyl alanine (for example, SEPIWHITE MSH available from Seppic); octadecenedioic acid (for example, ARLATONE DIOIC DCA available from Uniquema); oenothera biennis sead extract, and pyrus malus (apple) fruit extract, SMATVECTOR UV and Magnesium Ascorbyl Phosphate in Hyaluronic Filling Sphere available from COLETICA, and mixtures thereof.

Other skin active agents useful herein include those selected from the group consisting of N-acetyl-D-glucosamine (for example, N-acetyl-D-glucosamine available from Technical Sourcing International), panthenol (e.g., DL-Panthenol available from Alps Pharmaceutical Inc), tocopheryl nicotinate, benzoyl peroxide, 3-hydroxy benzoic acid, flavonoids (e.g., flavanone, chalcone), farnesol, phytantriol, glycolic acid, lactic acid, 4-hydroxy benzoic acid, acetyl salicylic acid, 2-hydroxybutanoic acid, 2-hydroxypentanoic acid, 2-hydroxyhexanoic acid, cis-retinoic acid, trans-retinoic acid, retinol, retinyl esters (e.g., retinyl propionate), phytic acid, N-acetyl-L-cysteine, lipoic acid, tocopherol (e.g., D-Delta-Tocopherol available from Eisai) and its esters (e.g., tocopheryl acetate: DL-α-tocopheryl acetate available from Eisai), azelaic acid, arachidonic acid, tetracycline, ibuprofen, naproxen, ketoprofen, hydrocortisone, acetominophen, resorcinol, phenoxyethanol, phenoxypropanol, phenoxyisopropanol, 2,4,4′-trichloro-2′-hydroxy diphenyl ether, 3,4,4′-trichlorocarbanilide, octopirox, lidocaine hydrochloride, clotrimazole, miconazole, ketoconazole, neomycin sulfate, theophylline, and mixtures thereof. In a preferred embodiment, the first layer of the present invention comprises from about 0.001% to about 30%, more preferably from about 0.001% to about 10% of a skin active agents.

Volatile Silicone Oil

The water-in-oil emulsion, oil-in-water emulsion or oil dispersion compositions of the present invention comprise volatile silicone oil, the pressed powder composition of the present invention comprises volatile silicone oil as an optional component. In a cosmetic foundation embodiment, the water-in-oil emulsion composition comprises from about 10% to about 50%, preferably from about 15% to about 35% of volatile silicone oil, oil-in-water emulsion composition comprises from about 1% to about 30%, preferably from about 5% to about 20% of volatile silicone oil and oil dispersion composition comprises from about 10% to about 80%, preferably from about 20% to about 70% of volatile silicone oil, when present, the pressed powder composition comprises from about 0.1% to about 10%, preferably from about 1% to about 5% of volatile silicone oil.

Without being bound by theory, the species and levels of the volatile silicone oil herein is believed to provide improved refreshing and light feeling to the skin, without necessarily leaving a dried feeling to the skin.

The volatile silicone oil useful herein are selected from those having a boiling point of from about 60 to about 260° C., preferably those having from 2 to 7 silicon atoms. The volatile silicone oils useful herein include polyalkyl or polyaryl siloxanes with the following structure (I): 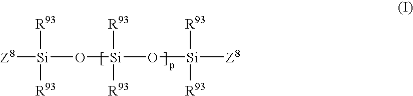
wherein R⁹³ is independently alkyl or aryl, and p is an integer from about 0 to about 5. Z⁸ represents groups which block the ends of the silicone chains. Preferably, R⁹³ includes methyl, ethyl, propyl, phenyl, methylphenyl and phenylmethyl, Z⁸ includes hydroxy, methyl, methoxy, ethoxy, propoxy, and aryloxy. More preferably, R⁹³ and Z⁸ are methyl. The preferred volatile silicone compounds are hexamethyldisiloxane, octamethyltrisiloxane, decamethyltetrasiloxane, hexadecamethylheptasiloxane. Commercially available volatile silicone compounds useful herein include octamethyltrisiloxane with tradename SH200C-1cs, decamethyltetrasiloxane with tradename SH200C-1.5cs, hexadecamethylheptasiloxane with tradename SH200C-2cs, all available from Dow Coming.

The volatile silicone oils useful herein also include a cyclic silicone compound having the formula: 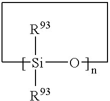
wherein R⁹³ is independently alkyl or aryl, and n is an integer of from 3 to 7.

Preferably, R⁹³ includes methyl, ethyl, propyl, phenyl, methylphenyl and phenylmethyl. More preferably, R⁹³ is methyl. The preferred volatile silicone compounds are octamethylcyclotetrasiloxane, decamethylcyclopentasiloxane, tetradecamethylcyclohexasiloxane. Commercially available volatile silicone compounds useful herein include octamethylcyclotetrasiloxane with tradename SH244, decamethylcyclopentasiloxane with tradename DC245 and SH245, and dodeamethylcyclohexasiloxane with tradename DC246; all available from Dow Coming.

Non-Volatile Oil

The water-in-oil emulsion, oil-in-water emulsion and oil dispersion compositions of the present invention comprise non-volatile oil. In a cosmetic foundation embodiment, the water-in-oil emulsion composition comprises from about 0.5% to about 20%, preferably from about 1% to about 15% of non-volatile oil, the oil-in-water emulsion composition comprises from about 1% to about 20%, preferably from about 5% to about 15% of non-volatile oil, and the oil dispersion composition comprises from about 1% to about 40%, preferably from about 5% to about 25% of non-volatile oil. Without being bound by theory, the species and levels of the non-volatile oil herein is believed to provide improved smoothness to the skin, and also alleviate dry feeling of the skin.

The pressed powder composition of the present invention comprises non-volatile oil as binder oil. In a cosmetic foundation embodiment, the pressed powder composition comprises from about 2% to about 40%, preferably from about 5% to about 25% of non-volatile oil. Without being bound by theory, the species and levels of the non-volatile oil herein is believed to provide improved smoothness to the skin, and also alleviate dry feeling of the skin.

Non-volatile oils useful herein are, for example, tridecyl isononanoate, isostearyl isostearate, isocetyl isosteatrate, isopropyl isostearate, isodecyl isonoanoate, cetyl octanoate, isononyl isononanoate, diisopropyl myristate, isocetyl myristate, isotridecyl myristate, isopropyl myristate, isostearyl palmitate, isocetyl palmitate, isodecyl palmitate, isopropyl palmitate, octyl palmitate, caprylic/capric acid triglyceride, glyceryl tri-2-ethylhexanoate, neopentyl glycol di(2-ethyl hexanoate), diisopropyl dimerate, tocopherol, tocopherol acetate, avocado oil, camellia oil, turtle oil, macadamia nut oil, corn oil, mink oil, olive oil, rapeseed oil, eggyolk oil, sesame oil, persic oil, wheat germ oil, pasanqua oil, castor oil, linseed oil, safflower oil, cotton seed oil, perillic oil, soybean oil, peanut oil, tea seed oil, kaya oil, rice bran oil, china paulownia oil, Japanese paulownia oil, jojoba oil, rice germ oil, glycerol trioctanate, glycerol triisopalmiatate, trimethylolpropane triisostearate, isopropyl myristate, glycerol tri-2-ethylhexanoate, pentaerythritol tetra-2-ethylhexanoate, lanolin, liquid lanolin, liquid paraffin, squalane, vaseline, and mixtures thereof. Commercially available oils include, for example, isotridecyl isononanoate with tradename Crodamol TN available from Croda, Hexalan available from Nisshin Seiyu, and tocopherol acetates available from Eisai.

Non-volatile oils useful herein also include polyalkyl or polyaryl siloxanes with the following structure (I) 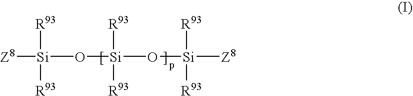
wherein R⁹³ is alkyl or aryl, and p is an integer from about 7 to about 8,000. Z⁸ represents groups which block the ends of the silicone chains. The alkyl or aryl groups substituted on the siloxane chain (R⁹³) or at the ends of the siloxane chains Z⁸ can have any structure as long as the resulting silicone remains fluid at room temperature, is dispersible, is neither irritating, toxic nor otherwise harmful when applied to the skin, is compatible with the other components of the composition, and is chemically stable under normal use and storage conditions. Suitable Z⁸ includes hydroxy, methyl, methoxy, ethoxy, propoxy, and aryloxy. The two R⁹³ groups on the silicon atom may represent the same group or different groups. Preferably, the two R93 groups represent the same group. Suitable R⁹³ includes methyl, ethyl, propyl, phenyl, methylphenyl and phenylmethyl. The preferred silicone compounds are polydimethylsiloxane, polydiethylsiloxane, and polymethylphenylsiloxane. Polydimethylsiloxane, which is also known as dimethicone, is especially preferred. The polyalkylsiloxanes that can be used include, for example, polydimethylsiloxanes. These silicone compounds are available, for example, from the General Electric Company in their Viscasil® and SF 96 series, and from Dow Coming in their Dow Coming 200 series such as SH 200.

Polyalkylaryl siloxane fluids can also be used and include, for example, polymethylphenylsiloxanes. These siloxanes are available, for example, from the General Electric Company as SF 1075 methyl phenyl fluid or from Dow Coming as 556 Cosmetic Grade Fluid or KF-56 from Shin-Etsu Chemical Co., Ltd.

Non-volatile oils also useful herein are the various grades of mineral oils. Mineral oils are liquid mixtures of hydrocarbons that are obtained from petroleum. Specific examples of suitable hydrocarbons include paraffin oil, mineral oil, dodecane, isododecane, hexadecane, isohexadecane, eicosene, isoeicosene, tridecane, tetradecane, polybutene, polyisobutene, and mixtures thereof.

Solid Wax

The water-in-oil emulsion and oil dispersion compositions of the present invention comprise a solid wax. In a cosmetic foundation embodiment, the water-in-oil emulsion composition comprises from about 1% to about 10%, preferably from about 2% to about 5% of solid wax; and the oil dispersion composition comprises from about 1% to about 10%, preferably from about 2% to about 7% of solid wax. Without being bound by theory, the species and levels of the solid wax herein is believed to provide consistency to the composition and coverage to the skin, while not negatively contributing to the spreadability upon application to the skin, and fresh and light feel of the skin.

The solid waxes useful herein are paraffin wax, microcrystalline wax, ozokerite was, ceresin wax, carnauba wax, candellila wax, eicosanyl behenate, and mixtures thereof. A mixture of waxes is preferably used.

Commercially available solid waxes useful herein include: Candelilla wax NC-1630 available from Cerarica Noda, Ozokerite wax SP-1021 available from Strahl & Pitsh, and Eicosanyl behenate available from Cas Chemical.

Lipophilic Surfactant

The water-in-oil emulsion composition of the present invention comprises a lipophilic surfactant, the oil dispersion composition and pressed powder composition may comprise lipophilic surfactant as an optional component. In a cosmetic foundation embodiment, the water-in-oil emulsion composition comprises from about 0.5% to about 5%, preferably from about 1% to about 4% of lipophilic surfactant, when present, the pressed powder composition comprises from about 0.1% to about 7%, preferably from about 0.3% to about 5% of lipophilic surfactant. The lipophilic surfactant herein has an HLB value of less than about 8.

The HLB value is a theoretical index value which describes the hydrophilicity-hydrophobicity balance of a specific compound. Generally, it is recognized that the HLB index ranges from 0 (very hydrophobic) to 40 (very hydrophilic). The HLB value of the lipophilic surfactants may be found in tables and charts known in the art, or may be calculated with the following general equation: HLB=7+(hydrophobic group values)+(hydrophilic group values). The HLB and methods for calculating the HLB of a compound are explained in detail in Surfactant Science Series, Vol. 1: Nonionic Surfactants”, pp 606-13, M. J. Schick (Marcel Dekker Inc., New York, 1966).

Without being bound by theory, the species and levels of the lipophilic surfactant herein are believed to provide a stable water-in-oil emulsion in view of the other components of the present invention.

The lipophilic surfactant can be an ester-type surfactant. Ester-type surfactants useful herein include: sorbitan monoisostearate, sorbitan diisostearate, sorbitan sesquiisostearate, sorbitan monooleate, sorbitan dioleate, sorbitan sesquioleate, glyceryl monoisostearate, glyceryl diiostearate, glyceryl sesquiisostearate, glyceryl monooleate, glyceryl dioleate, glyceryl sesquioleate, diglyceryl diisostearate, diglyceryl dioleate, diglycerin monoisostearyl ether, diglycerin diisostearyl ether, and mixtures thereof.

Commercially available ester-type surfactants are, for example, sorbitan isostearate having a tradename Crill 6 available from Croda, and sorbitan sesquioleate with tradename Arlacel 83 available from Kao Atras.

The lipophilic surfactant can be a silicone-type surfactant. Silicone-type surfactants useful herein are (i), (ii), and (iii) as shown below, and mixtures thereof.
(i) dimethicone copolyols having the formula: 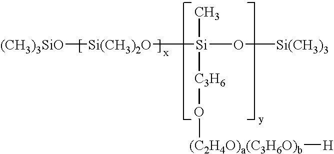
wherein x is an integer from 5 to 100, y is an integer from 1 to 50, a is zero or greater, b is zero or greater, the average sum of a+b is 1-100.
(ii) dimethicone copolyols having the formula: 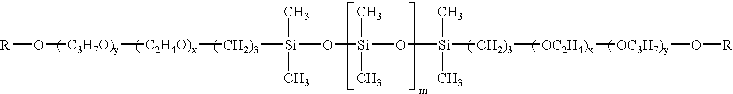
wherein R is selected from the group consisting of hydrogen, methyl, and combinations thereof, m is an integer from 5 to 100, x is independently zero or greater, y is independently zero or greater, the sum of x+y is 1-100.
(iii) branched polyether-polydiorganosiloxane emulsifiers herein having the formula: 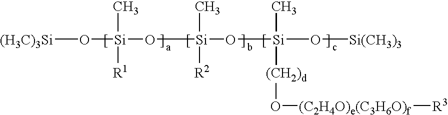
wherein R¹ is an alkyl having from about 1 to about 20 carbons; R² is 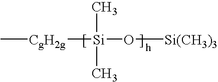
wherein g is from about 1 to about 5, and h is from about 5 to about 20; R³ is H or an alkyl having from about 1 to about 5 carbons; e is from about 5 to about 20; f is from about 0 to about 10; a is from about 20 to about 100; b is from about 1 to about 15; c is from about 1 to about 15; and d is from about 1 to about 5.

Commercially available silicone-type surfactants are, for example, dimethicone copolyols DC5225C, BY22-012, BY22-008, SH3746M, SH3771M, SH3772M, SH3773M, SH3775M, SH3748, SH3749, and DC5200, all available from Dow Coming, and branched polyether-polydiorganosiloxane emulsifiers such as PEG-9 polydimethylsiloxyethyl Dimethicone, having an HLB of about 4 and a molecular weight of about 6,000 having a tradename KF-6028 available from ShinEtsu Chemical.

In a preferred embodiment, the lipophilic surfactant is a mixture of at least one ester-type surfactant and at least one silicone-type surfactant to provide a stable emulsion for the other essential components of the present invention.

Water

The water-in-oil emulsion and oil-in-water emulsion compositions of the present invention comprise water in an amount of sufficient to provide a discontinuous or continuous aqueous phase. In a cosmetic foundation embodiment, the water-in-oil emulsion composition comprises an amount of water such that the total level of volatile silicone oil and water is more that about 40%, preferably from about 10% to about 35% of water; and the oil-in-water emulsion composition comprises from about 20% to about 60%, preferably from about 30% to about 50% of water. Without being bound by theory, the amount of water herein is believed to provide improved refreshing and light feeling to the skin, without necessarily leaving a dried feeling to the skin. Further, this amount of water allows the inclusion of optional water-soluble skin active agents as described below.

In the present invention, deionized water is typically used. Water from natural sources including mineral cations can also be used, depending on the desired characteristic of the product.

Fatty Compounds and Fatty Acid Salts

The oil-in-water emulsion composition of the present invention comprises fatty compounds or fatty acid salt. In a cosmetics foundation embodiment, the oil-in-water emulsion composition comprises from about 1% to about 15%, more preferably from about 2% to about 10% of fatty compounds or fatty acid salt.

Fatty compounds and Fatty acid salt useful herein include stearic acid (e.g., Stearic Acid 750 available from Kao), staric acid sodium salt, palmitic acid, stearyl alcohol, cetyl alcohol, behenyl alcohol, stearic acid, palmitic acid, the polyethylene glycol ether of stearyl alcohol or cetyl alcohol having an average of about I to about 5 ethylene oxide units, and mixtures thereof. Preferred fatty compounds are selected from stearyl alcohol, cetyl alcohol, behenyl alcohol, the polyethylene glycol ether of stearyl alcohol having an average of about 2 ethylene oxide units (steareth-2), the polyethylene glycol ether of cetyl alcohol having an average of about 2 ethylene oxide units, and mixtures thereof.

Hydrophilic Surfactant

The oil-in-water emulsion composition of the present invention comprises hydrophilic surfactant. In a cosmetic foundation embodiment, the oil-in-water emulsion composition comprises from about 0.1% to about 4%, more preferably from about 0.3% to about 2% of hydrophilic surfactant.

A wide variety of hydrophilic surfactant can be employed herein. Known or conventional hydrophilic surfactant can be used in the composition, provided that the selected hydrophilic surfactant is chemically and physically compatible with essential components of the composition, and provides the desired dispersion characteristics.

Non-limiting examples of hydrophilic surfactant useful herein are various non-ionic and anionic hydrophilic surfactant such as sugar esters and polyesters, alkoxylated sugar esters and polyesters, C1-C30 fatty acid esters of C1-C30 fatty alcohols, alkoxylated derivatives of C1-C30 fatty acid esters of C1-C30 fatty alcohols, alkoxylated ethers of C1-C30 fatty alcohols, polyglyceryl esters of C1-C30 fatty acids, C1-C30 esters of polyols, C1-C30 ethers of polyols, alkyl phosphates, polyoxyalkylene fatty ether phosphates, fatty acid amides, acyl lactylates, soaps, polyoxyalkylene hydrogenated caster oils, polyglycerin alkyl esters having the C10-20 of alkylsubstitute, polyoxyethylene sterols, and polyoxyethylene hydrogenated sterols and mixtures thereof.

Non-limiting examples of other hydrophilic surfactant for use herein include: polyethylene glycol 20 sorbitan monolaurate (polysorbate 20), polyethylene glycol 5 soya sterol, steareth-20, ceteareth-20, PPG-2 methyl glucose ether distearate, ceteth- 10, polysorbate 80, cetyl phosphate, potassium cetyl phosphate, diethanolamine cetyl phosphate, polysorbate 60, glyceryl stearate, PEG-100 stearate, polyoxyethylene 20 sorbitan trioleate (polysorbate 85), sorbitan monolaurate, polyoxyethylene 4 lauryl ether sodium stearate, polyglyceryl-4 isostearate, hexyl laurate, PPG-2 methyl glucose ether distearate, ceteth- 10, diethanolamine cetyl phosphate, glyceryl stearate, PEG 40 hydrogenated castor oil, PEG-60 hydrogenated castor oil, and mixtures thereof.

Polyoxyalkylene hydrogenated castor oils useful herein include, for example, polyoxyethylene hydrogenated castor oils having 20-100 moles of ethylene oxides, such as polyoxyethylene (20) hydrogenated castor oil, polyethylene (40) hydrogenated castor oil, and polyoxyethylene (100) hydrogenated castor oil.

Polyglycerin alkyl esters having the C10-20 of alkylsubstitute useful herein include,-for example, those having 6-10 moles of glycerin units, such as polyglyceryl-6 laurate, polyglyceryl-10 laurate, and polyglyceryl-10 stearate.

Polysorbates useful herein include, for example, those having 20-80 moles of ethylene oxides, such as polysorbate-20, polyborbate-40, polysorbate-60, and polysorbate-80.

Polyethylene sterols and polyethylene hydrogenated sterols useful herein include, for example, those having 10-30moles of ethylene oxides, such as polyethylene (10) phytosterol, polyethylene (30) phytosterol, and polyethylene (20) cholesterol.

Among the above nonionic surfactants, preferred are polysorbates, and more preferred are polysorbate-20, polysorbate-40, and mixtures thereof.

Commercially available hydrophilic surfactant is glyceryl stearate : Arlacel 161 available from Uniqema.

Pigment Powder Component

Compositions of pressed powder, water-in-oil emulsion, and oil-in-water emulsion of the present invention comprises powder component. In a cosmetic foundation embodiment, the water-in-oil emulsion composition comprises from about 5% to about 45%, more preferably from about 15% to about 30% of powder component, the oil-in-water emulsion composition comprises from about 5% to about 40%, more preferably from about 15% to about 30% of pigment powder component, and the pressed powder composition comprises from about 60% to about 98%, more preferably from about 75% to about 95% of powder component. The oil dispersion composition optionally comprises a pigment powder, when present; the content level of pigment powder in the oil dispersion composition is from about 1% to about 70%, more preferably from about 5% to about 50%. The pigment powder used herein is typically hydrophobic in nature, or hydrophobically treated for water-in-oil emulsion and oil dispersion, and hydrophilic in nature or non-hydrophobically treated for oil-in-water emulsion. Both hydrophobic and hydrophilic pigment powder components can be used for pressed powder.

By keeping the level of pigment component low, the entire composition maintains flexibility to accommodate other components which provide spreadability, moisturization, and fresh and light feel.

The species and levels of the pigments are selected to provide, for example, shade, coverage, good wear performance, and stability in the composition.

Pigments are useful for the pigment component herein are inorganic and organic powders such as talc, mica, sericite, silica, magnesium silicate, synthetic fluorphlogopite, calcium silicate, aluminum silicate, bentonite and montmorillonite; pearl pigments such as alumina, barium sulfate, calcium secondary phosphate, calcium carbonate, titanium oxide, finely divided titanium oxide, zirconium oxide, zinc oxide, hydroxy apatite, iron oxide, iron titanate, ultramarine blue, Prussian blue, chromium oxide, chromium hydroxide, cobalt oxide, cobalt titanate, titanium oxide coated mica; organic powders such as polyester, polyethylene, polystyrene, methyl methacrylate resin, cellulose, 12-nylon (e.g., NYLON POWDER SP-500 available from TORAY), 6-nylon, styrene-acrylic acid copolymers, polypropylene, vinyl chloride polymer, tetrafluoroethylene polymer, boron nitride, fish scale guanine, laked tar color dyes, and laked natural color dyes. Such pigments may be treated with a hydrophobical treatment agent, including: silicone such as Methicone, Dimethicone, and perfluoroalkylsilane; fatty material such as stearic acid and disodium hydrogenated glutamate; metal soap such as aluminium dimyristate; aluminium hydrogenated tallow glutamate, hydrogenated lecithin, lauroyl lysine, aluminium salt of perfluoroalkyl phosphate, and aluminium hydroxide as to reduce the activity for titanium dioxide, and mixtures thereof.

Commercially available hydrophobic pigment powder components include coverage titanium dioxide, such as titanium dioxide and talc and methicone: SI-T-CR-50Z available, titanium dioxide and methicone: SI-Titanium Dioxide IS, titanium dioxide and dimethicone: SA-Titanium Dioxide CR-50, titanium dioxide and methicone: SI-FTL-300 and titanium dioxide and dimethicone and disodium hydrogenated glutamate: SA/NAI-TR-10, all of them are available from Miyoshi Kasei, iron oxide and cyclopentasiloxane and disodium hydrogenated glutamate: SA/NAI-Y-10/D5(70%)/SA/NAI-R-10/D5(65%) /SA/NAI-B-10/D5(75%) available from Miyoshi Kasei, iron oxide and disodium hydrogenated glutamate: SA/NAI-Y-10/SA/NAI-R-10/SA/NAI-B-10 available from Miyoshi Kasei, iron oxide and methicone: SI Mapico Yellow Light Lemon XLO/SI Pure Red Iron Oxide R-1599/SI Pure Red Iron Oxide R-3098/SI Pure Red Iron Oxide R-4098/SI Black Iron Oxide No.247 available from Daito Kasei, alumina and titanium dioxide and methicone: SI-LTSG30AFLAKE H (5%) LHC available from Miyoshi Kasei, talc and methicone: SI-Talc CT-20 available from Miyoshi Kasei, talc and methicone: SI-Talc JA13R LHC available from Miyoshi Kasei, mica and methicone: SI Mica available from Miyoshi Kasei, silica and dimethicone: SA-SB-300 available from Miyoshi Kasei, mica and methicone: SI Sericite available from Miyoshi Kasei, mica and dimethicone: SA Sericite available from Miyoshi Kasei, mica and C9-15 fluoroalcol phosphates and triethoxy caprylylsilane: FOTS-52 Sericite FSE available from Daito Kasei, talc and C9-15 fluoroalcol phosphates and triethoxy caprylylsilane: FOTS-52 Talc JA-13R available from Daito Kasei, boron nitride and methicone: SI02 Boron Nitride SHP-6 available from Daito Kasei, boron nitride and C9-15 fluoroalcol phosphates and triethoxy caprylylsilane: FOTS-52 Boron Nitride available from Daito Kasei, mica and titanium dioxide and methicone: SI Sericite TI-2 available from Miyoshi Kasei, mica and titanium dioxide and methicone: SI Mica TI-2 available from Miyoshi Kasei, talc and titanium dioxide and methicone: SI Talc TI-2 available from Miyoshi Kasei, lauroyl lysine: AMIHOPE LL available from Ajinomoto, synthetic fluorphlogopite and methicone: PDM-5L(S) / PDM-10L(S)/PDM-20L(S)/PDM-40L(S) available from Topy Industries,

Commercially available hydrophilic pigment components include coverage titanium dioxide, such as Titanium dioxide CR-50 available from Ishihara Techno Corporation, mica: Mica Y-3000 available from Yamaguchi Mica, talc: Talc JA13R available from Asada Milling, silica: MK-30 available from Fuji Silysia, iron oxides available from Titan Kogyo, boron nitride: Boron Nitride SHP-6 available from Mizushima Ferroalloy, barium sulfate: Pletelet Barium sulfate H, HF, HG, HL, HM, HP available from Sakai Chemical Industry.

Additional Components

The compositions hereof may further contain additional components such as are conventionally used in topical products, e.g., for providing aesthetic or functional benefit to the composition or skin, such as sensory benefits relating to appearance, smell, or feel, therapeutic benefits, or prophylactic benefits (it is to be understood that the above-described required materials may themselves provide such benefits).

The CTFA Cosmetic Ingredient Handbook, Second Edition (1992) describes a wide variety of nonlimiting cosmetic and pharmaceutical ingredients commonly used in the industry, which are suitable for use in the topical compositions of the present invention. Such other materials may be dissolved or dispersed in the composition, depending on the relative solubilities of the components of the composition.

Examples of suitable topical ingredient classes include: anti-cellulite agents, antioxidants, radical scavengers, chelating agents, vitamins and derivatives thereof, abrasives, other oil absorbents, astringents, dyes, essential oils, fragrance, structuring agents, emulsifiers, solubilizing agents, anti-caking agents, antifoaming agents, binders, buffering agents, bulking agents, denaturants, pH adjusters, propellants, reducing agents, sequestrants, cosmetic biocides, and preservatives.

Humectant

The composition of the present invention may further comprise a humectant at from about 1% to about 15%, preferably 2% to about 7% of each layer.

The humectants herein are selected from the group consisting of polyhydric alcohols, water soluble alkoxylated nonionic polymers, and mixtures thereof. Polyhydric alcohols useful herein include glycerin, propylene glycol, 1,3-butylene glycol, dipropylene glycol, diglycerin, sodium hyaluronate, and mixtures thereof.

Commercially available humectants herein include: glycerin available from Asahi Denka; propylene glycol with tradename LEXOL PG-865/855 available from Inolex, 1-,2-PROPYLENE GLYCOL USP available from BASF; 1,3-butylene glycol available from Kyowa Hakko Kogyo; dipropylene glycol with the same tradename available from BASF; diglycerin with tradename DIGLYCEROL available from Solvay GmbH; sodium hyaluronate with tradenames ACTIMOIST available from Active Organics, AVIAN SODIUM HYALURONATE series available from Intergen, HYALURONIC ACID Na available from Ichimaru Pharcos.

UV Absorbing Agent

The compositions of the present invention may comprise a safe and effective amount of a UV absorbing agent. A wide variety of conventional UV protecting agent are suitable for use herein, such as those described in U.S. Pat. No. 5,087,445, Haffey et al, issued Feb. 11, 1992; U.S. Pat. No. 5,073,372, Turner et al, issued Dec. 17, 1991; U.S. Pat. No. 5,073,371, Turner et al., issued Dec. 17, 1991; and Segarin, et al, at Chapter VIII, pages 189 et seq., of Cosmetics Science and Technology (1972). When included, the present composition comprises from about 0.5% to about 20%, preferably from about 1% to about 15% of a UV absorbing agent.

UV absorbing agents useful herein are, for example, 2-ethylhexyl-p-methoxycinnamate (commercially available as PARSOL MCX), butylmethoxydibenzoyl-methane, 2-hydroxy-4-methoxybenzo-phenone, 2-phenylbenzimidazole-5-sulfonic acid, octyldimethyl-p-aminobenzoic acid, octocrylene, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyldibenzoylmethane, 4-isopropyl dibenzoylmethane, 3-benzylidene camphor, 3-(4-methylbenzylidene) camphor, Eusolex™ 6300, Octocrylene, Avobenzone (commercially available as Parsol 1789), and mixtures thereof.

UV Protection Powder

The composition of each layer of the present invention comprises from about 0% to about 20%, preferably from about 0.1% to about 10% of a UV protection powders, such as micronized titanium dioxide and micronized zinc oxide. The powders included in the pigment component herein are typically hydrophobic in nature, or hydrophobically treated. The species and levels of the powders to provide UV protection benefit in the composition. The particle size of UV protection powders is less than 100 nm which size does not have coverage effects.

Commercially available UV protection powders are titanium dioxide and methicone SI-TTO-S-3Z available from Miyoshi Kasei, titanium dioxide and dimethicone and aluminum hydroxide and stearic acid: SAST-UFTR-Z available from Miyoshi Kasei, zinc oxide: Finex series available from Sakai Chemical Industry.

Thickener

Useful for the present invention is a thickener. Thickeners can be used for solidifying the compositions of the present invention. When used, the thickener is kept to about 15% of the entire composition. The thickeners useful herein include gelling agents, inorganic thickeners, and mixtures thereof. The amount and type of thickeners are selected according to the desired viscosity and characteristics of the product.

The gelling agents useful as thickeners of the present invention include esters and amides of fatty acid gellants, hydroxy acids, hydroxy fatty acids, other amide gellants, and crystalline gellants.

N-acyl amino acid amides useful herein are prepared from glutamic acid, lysine, glutamine, aspartic acid and mixtures thereof. Particularly preferred are n-acyl glutamic acid amides corresponding to the following formula:
R2-NH—CO—(CH2)2-CH—(NH—CO—R1)—CO—NH—R2
wherein R1 is an aliphatic hydrocarbon radical having from about 12 to about 22 carbon atoms, and R2 is an aliphatic hydrocarbon radical having from about 4 to about 12 carbon atoms. Non-limiting examples of these include n-lauroyl-L-glutamic acid dibutyl arnide, n-stearoyl-L-glutamic acid diheptyl amide, and mixtures thereof. Most preferred is n-lauroyl-L-glutamic acid dibutyl amide, also referred to as dibutyl lauroyl glutamide. This material is commercially available with tradename Gelling agent GP-1 available from Ajinomoto.

Other gelling agents suitable for use in the compositions include 12-hydroxystearic acid, esters of 12-hydroxystearic acid, amides of 12-hydroxystearic acid and combinations thereof. These preferred gellants include those which correspond to the following formula:
R1-CO—(CH2)10-CH—(OH)—(CH2)5-CH3
wherein R1 is R2 or NR2R3; and R2 and R3 are hydrogen, or an alkyl, aryl, or arylalkyl radical which is branched linear or cyclic and has from about 1 to about 22 carbon atoms; preferably, from about 1 to about 18 carbon atoms. R2 and R3 may be either the same or different; however, at least one is preferably a hydrogen atom. Preferred among these gellants are those selected from the group consisting of 12-hydroxystearic acid, 12-hydroxystearic acid methyl ester, 12-hydroxystearic acid ethyl ester, 12-hydroxystearic acid stearyl ester, 12-hydroxystearic acid benzyl ester, 12-hydroxystearic acid amide, isopropyl amide of 12-hydroxystearic acid, butyl amide of 12-hydroxystearic acid, benzyl amide of 12-hydroxystearic acid, phenyl amide of 12-hydroxystearic acid, t-butyl arnide of 12-hydroxystearic acid, cyclohexyl amide of 12-hydroxystearic acid, 1-adamantyl amide of 12-hydroxystearic acid, 2-adamantyl amide of 12-hydroxystearic acid, diisopropyl amide of 12-hydroxystearic acid, and mixtures thereof; even more preferably, 12-hydroxystearic acid, isopropyl amide of 12-hydroxystearic acid, and combinations thereof. Most preferred is 12-hydroxystearic acid.

Suitable amide gellants include disubstituted or branched monoamide gellants, monosubstituted or branched diamide gellants, triamide gellants, and combinations thereof, excluding the n-acyl amino acid derivatives selected from the group consisting of n-acyl amino acid amides, n-acyl amino acid esters prepared from glutamic acid, lysine, glutamine, apartic acid, and combinations thereof, and which are specifically disclosed in U.S. Pat. No. 5,429,816.

Alkyl amides or di- and tri-basic carboxylic acids or anhydrides suitable for use in the composition include alkyl amides of citric acid, tricarballylic acid, aconitic acid, nitrilotriacetic acid, succinic acid and itaconic acid such as 1,2,3-propane tributylamide, 2-hydroxy-1,2,3-propane tributylamide, 1-propene-1,2,3-triotylamide, N,N′,N″-tri(acetodecylamide)amine, 2-dodecyl-N,N′-dihexylsuccinamide, and 2 dodecyl-N,N′-dibutylsuccinamide. Preferred are alkyl amides of di-carboxylic acids such as di-amides of alkyl succinic acids, alkenyl succinic acids, alkyl succinic anhydrides and alkenyl succinic anhydrides, more preferably 2-dodecyl-N,N′-dibutylsuccinamide.

Inorganic thickeners useful herein include hectorite, bentonite, montmorillonite, and bentone clays which have been modified to be compatible with oil. Preferably, the modification is quaternization with an ammonium compound. Preferable inorganic thickeners include quaternary ammonium modified hectorite. Commercially available oil swelling clay materials include benzyldimethyl stearyl ammonium hectorite with tradename Bentone 38 available from Elementis.

Preparation of the Composition

While the present composition may be made by any process known in the art, the process herein is advantageous for manufacturing the present composition in an aesthetically appealing, yet cost effective manner.

The present process is particularly useful for the present composition wherein the first layer has a viscosity of from about 100 mPas to about 10,000 mPas, preferably from 300 mPas to 3000 mPas when brought to a temperature of between about 55° C. and about 90° C. The present process comprises the steps of:

(a) providing the first layer composition selected from water-in-oil emulsion, oil-in-water emulsion and oil dispersion in fluid state and the second layer composition in pulverized powder state in isolated vessels;

(b) dispensing a predetermined amount of the pulverized powder bulk composition into a primary package, such as a pan, and then pressing the bulk powder by a conventional pressing machine;

(c) dispensing the first layer composition into the remaining part of the same primary package of step (b) while keeping the temperature of the first layer composition at between 55° C. and 90° C., preferably between 60° C. and 75° C.; and

(d) allowing the transferred first layer composition to solidify in the package.

Each of the first and second layer compositions can be made by any suitable method known for providing water-in-oil emulsion composition, oil-in-water emulsion composition, oil dispersion composition and powder composition. In a suitable process, the first and second layer compositions are independently made by the steps of:

1. Powder Composition

In a suitable process, the powder composition is made by the steps of:
1) Mixing and pulverizing all powder materials to prepare a powder mixture;
2) Dissolving the non-volatile oils to make a binder base at about 80° C.;
3) Adding the binder base of step 2) into the powder mixture of step 1) and mixing until well dispersed; and
4) Pulverizing the product of step 3)
   2. Oil Dispersion Composition

In a suitable process, the oil dispersion composition is made by the steps of:
1) Dissolving the volatile silicone oil, non-volatile oil, pigments slurry dispersed in oil, and any other hydrophobic material in liquid form at ambient temperature in a sealed tank, to make a lipophilic mixture;
2) Adding the remaining pigments and powders into such lipophilic mixture and dispersing with a homogenizer at about 20-30° C.;
3) Heating the lipophilic mixture of step 2) and adding solid wax and any remaining hydrophobic material at about 80-85° C.; and
4) Cooling the finally obtained emulsion to a temperature of about 60-80° C.

The obtained composition, which is still fluid at such temperature, is filled into an air-tight container and allowed to cool to room temperature typically using a cooling unit. The obtained composition is solid at ambient temperature, and thus can be poured into such container and left to solidify.

3. Oil-in-water Emulsion Composition

In a suitable process, the oil-in-water emulsion composition is made by the steps of:
1) Dissolving water, humentant, fatty acid salts and any other hydrophilic material in liquid form at about 80-85° C. in a sealed tank, to make a hydrophilic mixture;
2) Adding the remaining pigments and powders into such hydrophilic mixture and dispersing with a homogenizer;
3) Dissolving the volatile silicone oil, non-volatile oil, fatty compound in oil, and any other hydrophobic material in liquid form at ambient temperature in a sealed tank, to make a lipophilic mixture;
4) Adding the lipophilic mixture of step 3) to the hydrophilic mixture of step 2) to effect an emulsification;
5) Cooling the finally obtained emulsion to a temperature of about 60-80° C.

The obtained composition, which is still fluid at such temperature, is filled into an air-tight container and allowed to cool to room temperature typically using a cooling unit. The obtained composition is solid at ambient temperature, and thus can be poured into such container and left to solidify.

4. Water-in-oil Emulsion Composition

In a suitable process, the water-in-oil emulsion composition is made by the steps of:
1) Dissolving the volatile silicone oil, non-volatile oil, lipophilic surfactant, slurry of pigments dispersed in oil, and any other hydrophobic material in liquid form at ambient temperature in a sealed tank, to make a lipophilic mixture;
2) Adding the remaining pigments and powders into such lipophilic mixture and dispersing with a homogenizer at about 20-30° C.;
3) Separate from 1) and 2), heating and dissolving in water, humectants and any other hydrophilic material at about 75-80° C., and then cooling to about 20-30° C.;
4) Adding the product of step 3) to the product of step 2) to effect an emulsification;
5) Heating and adding solid wax and any remaining hydrophobic material into the product of step 4) at about 80-85° C.; and
6) Cooling the finally obtained emulsion to a temperature of about 60-80° C.

The obtained composition, which is still fluid at such temperature, is filled into an air-tight container and allowed to cool to room temperature typically using a cooling unit. The obtained composition is solid at ambient temperature, and thus can be poured into such container and left to solidify.

### EXAMPLES

The following examples further describe and demonstrate the preferred embodiments within the scope of the present invention. The examples are given solely for the purpose of illustration, and are not to be construed as limitations of the present invention since many variations thereof are possible without departing from its spirit and scope.

### 1) Examples 1-5

### W/O Solid Emulsion Layer

The following water-in-oil emulsion make-up compositions are formed by the process described below:

| No. | Components | Ex. 1-1 | Ex. 2-1 | Ex. 3-1 | Ex. 4-1 | Ex. 5-1 |
|---|---|---|---|---|---|---|
| 1 | Cyclopentasiloxane *1 | 25.90 | 25.90 | 25.90 | 15.90 | 16.90 |
| 2 | PEG-9 Polydimethylsiloxyethyl | 1.50 | 1.50 | 1.50 | 1.50 | 1.50 |
| | Dimethicone *2 | | | | | |
| 3 | Dimethicone and | — | — | — | — | 10.00 |
| | Dimethicone/Vinyl | | | | | |
| | Dimethicone Crosspolymer *3 | | | | | |
| 4 | Trimethylsiloxysilicate and | — | — | — | 10.00 | — |
| | Cyclopentasiloxane *4 | | | | | |
| 5 | Tocopheryl Acetate *5 | 0.50 | 0.50 | 0.50 | 0.50 | 0.50 |
| 6 | Isotridecyl Isononanoate *6 | 2.00 | 2.00 | 2.00 | 2.00 | 2.00 |
| 7 | Sorbitan Monoisostearate *7 | 1.50 | 1.50 | 1.50 | 1.50 | 1.50 |
| 8 | Iron oxide and | 2.00 | 1.80 | 2.00 | 2.00 | 2.00 |
| | Cyclopentasiloxane and | | | | | |
| | Dimethicone and Disodium | | | | | |
| | Hydrogenated Glutamate *8 | | | | | |
| 9 | Titanium Dioxide and | — | — | — | — | 8.00 |
| | Dimethicone and Disodium | | | | | |
| | Hydrogenated Glutamate *9 | | | | | |
| 10 | Titanium Dioxide and Talc and | 14.00 | 12.60 | 10.00 | 12.00 | — |
| | Methicone *10 | | | | | |
| 11 | Alumina and Titanium Dioxide | — | — | — | 2.00 | 3.00 |
| | and Methicone *11 | | | | | |
| 12 | Titanium Dioxide and | — | — | 5.00 | — | — |
| | Methicone *12 | | | | | |
| 13 | Titanium Dioxide and | 3.00 | 3.00 | 3.00 | 3.00 | 5.00 |
| | Dimethicone and Aluminium | | | | | |
| | Hydroxide and Stearic Acid *13 | | | | | |
| 14 | Talc and Methicone *14 | 7.00 | 8.60 | 6.00 | 7.00 | 7.00 |
| 15 | Water | 29.00 | 29.00 | 30.00 | 28.00 | 29.00 |
| 16 | Niacinamide *15 | 4.00 | 4.00 | 3.00 | 3.00 | 4.00 |
| 17 | N-acetyl D-glucosamine | — | — | — | 2.00 | — |
| 18 | Preservative | 0.45 | 0.45 | 0.45 | 0.45 | 0.45 |
| 19 | Panthenol *16 | 0.25 | 0.25 | 0.25 | 0.25 | 0.25 |
| 20 | Glycerin *17 | — | — | — | 2.00 | 5.00 |
| 21 | Butylene Glycol *18 | 5.00 | 5.00 | 5.00 | 3.00 | — |
| 22 | Candelilla Wax *19 | 2.00 | 2.00 | 2.00 | 2.00 | 2.00 |
| 23 | Ceresin *20 | 1.90 | 1.90 | 1.90 | 1.90 | 1.90 |

| | | | | | |
|---|---|---|---|---|---|
| Total | 100.00 | 100.00 | 100.00 | 100.00 | 100.00 |

Definitions of Components
*1 Cyclopentasiloxane: SH245 available from Dow Corning
*2 PEG-9 Polydimethylsiloxyethyl Dimethicone: KF-6028 available from Shin-Etsu Chemical Co., Ltd.
*3 Dimethicone and Dimethicone/Vinyl Dimethicone Crosspolymer: KSG-16 available from Shin-Etsu Chemical Co., Ltd.
*4 Trimethylsiloxysilicate and Cyclopentasiloxane: Trimethilsiloxysilicate/Cyclomethicone D5 Blend available from GE Toshiba Silicones
*5 Tocopheryl Acetate: DL-α-tocopheryl Acetate available from Eisai
*6 Isotridecyl isononanoate: Crodamol TN available from Croda
*7 Sorbitan monoisostearate: Crill 6 available from Croda
*8 Iron Oxide and Cyclopentasiloxane and Dimethicone and Disodium Hydrogenated Glutamate: SA/NAI-Y-10/D5 (70%), SA/NAI-R-10/D5 (65%) and SA/NAI-B-10/D5 (75%) available from Miyoshi Kasei
*9 Titanium Dioxide and Dimethicone and Disodium Hydrogenated Glutamate: SA/NAI TR-10 from Miyoshi Kasei
*10 Titanium Dioxide and Talc and Methicone: SI-T-CR-50Z available from Miyoshi Kasei
*11 Alumina and Titanium Dioxide and Methicone: SI-LTSG30AFLAKEH(5%)LHC available from Miyoshi Kasei
*12 Titanium Dioxide and Methicone: SI-FTL-300 available from Miyoshi Kasei
*13 Titanium Dioxide and Dimethicone and Aluminium Hydroxide and Stearic acid: SAST-UFTR-Z available from Miyoshi Kasei
*14 Talc and Methicone: SI Talc JA13R LHC available from Miyoshi Kasei
*15 Niacinamide: Niacinamide available from Reilly Industries Inc.
*16 Panthenol: DL-Panthenol available from Alps Pharmaceutical Inc.
*17 Glycerin: Glycerin USP available from Asahi Denka
*18 Butylene Glycol: 1,3 Butylene Glycol available from Kyowa Hakko Kogyo
*19 Candelilla wax: Candelilla wax NC-1630 available from Cerarica Noda
*20 Ceresin: Ozokerite wax SP-1021 available from Strahl & Pitsh

Preparation Method

The W/O solid emulsion composition of the first layer of Examples 1-1 -5-1 are prepared as follows:
1) Mixing components numbers 1 through 8 with a suitable mixer until homogeneous to make a silicone phase.
2) Mixing components numbers 9 through 14 with a suitable mixer until homogeneous to make a pigment mixture which is then pulverized using a pulverizer. Then adding the pigment mixture into the silicone phase with a suitable mixer until homogeneous.
3) Dissolving components number 15 through 21 with suitable mixer until all components are dissolved to make a water phase which is then added into the silicone phase and pigment mixture to make an emulsion at room temperature using homogenizer.
4) Adding components number 22 and 23 into the emulsion which is then heated to dissolve at 85° C. in a sealed tank.
5) Finally, filling the emulsion into an air-tight container and allowing cooling to room temperature using a cooling unit.

### 2) Examples 6-10

### O/W Solid Emulsion Layer

The following oil-in-water emulsion make-up compositions are formed by the process described herein:

| No. | Components | Ex. 6-1 | Ex. 7-1 | Ex. 8-1 | Ex. 9-1 | Ex. 10-1 |
|---|---|---|---|---|---|---|
| 1 | Cyclopentasiloxane *1 | 12.50 | 10.50 | 14.50 | 10.50 | 10.50 |
| 2 | Stearic Acid *2 | 1.20 | 1.20 | 1.20 | 1.20 | 1.20 |
| 3 | Glyceryl Stearate *3 | 1.00 | 1.00 | 1.00 | 1.00 | 1.00 |
| 4 | Tocopheryl Acetate *4 | 0.50 | 0.50 | 0.50 | 0.50 | 0.50 |
| 5 | Isotridecyl Isononanoate *5 | 2.00 | 2.00 | 2.00 | 2.00 | 2.00 |
| 6 | Phenyl Trimethicone *6 | 8.00 | 8.00 | 8.00 | 8.00 | 8.00 |
| 7 | Iron oxides | 2.00 | 2.00 | 2.00 | — | — |
| 8 | Titanium Dioxide *7 | 8.00 | 8.00 | 8.00 | — | — |
| 9 | Mica *8 | 5.00 | 5.00 | 5.00 | 7.00 | 7.00 |
| 10 | Talc *9 | 6.00 | 6.00 | 6.00 | 14.00 | 14.00 |
| 11 | Water | 35.76 | 39.76 | 35.00 | 35.76 | 35.00 |
| 12 | Stearic acid Sodium Salt | 6.50 | 6.50 | 6.50 | 6.50 | 6.50 |
| 13 | Triethanolamine | 0.10 | 0.10 | 0.10 | — | — |
| 14 | Potassium Hydroxide | — | — | — | 0.10 | 0.10 |
| 15 | Niacinamide *10 | 3.50 | 3.50 | 3.50 | 3.50 | 3.50 |
| 16 | Ascorbyl Glucoside *11 | 2.00 | 2.00 | — | 2.00 | — |
| 17 | Sodium Hydroxide | 0.24 | 0.24 | — | 0.24 | — |
| 18 | N-acetyl D-glucosamine *12 | 2.00 | — | — | 2.00 | 2.00 |
| 19 | Magnesium Ascorbyl | — | — | 3.00 | — | 3.00 |
| | Phosphate *13 | | | | | |
| 20 | Preservative | 0.45 | 0.45 | 0.45 | 0.45 | 0.45 |
| 21 | Panthenol *14 | 0.25 | 0.25 | 0.25 | 0.25 | 0.25 |
| 22 | Glycerin *15 | — | — | 3.00 | — | 2.00 |
| 23 | Butylene Glycol *16 | 3.00 | 3.00 | — | 5.00 | 3.00 |

| | | | | | |
|---|---|---|---|---|---|
| Total | 100.00 | 100.00 | 100.00 | 100.00 | 100.00 |

Definitions of Components
*1 Cyclopentasiloxane: SH245 available from Dow Corning
*2 Stearic Acid: Stearic Acid 750 available from Kao
*3 Glyceryl Stearate: Arlacel 161 available from Uniqema
*4 Tocopheryl Acetate: DL-α-tocopheryl Acetate available from Eisai
*5 Isotridecyl isononanoate: Crodamol TN available from Croda
*6 Phenyl Trimethicone: KF-56 available from Shin-Etsu Chemical Co., Ltd.
*7 Titanium Dioxide: Titanium Dioxide CR-50 available from Ishihara Techno Corporation
*8 Mica: Mica Y-3000 available from Yamaguchi Mica
*9 Talc: Talc JA13R available from Asada Milling
*10 Niacinamide: Niacinamide available from Reilly Industries Inc.
*11 Ascorbyl Glucoside: Ascorbyl Glucoside available from Hayashibara
*12 N-acetyl D-glucosamine: N-acetyl D-glucosamine available from Technical Sourcing International
*13 Magunesium Ascorbyl Phosphate: Magunesium Ascorbyl Phosphate available from Showa Denko
*14 Panthenol: DL-Panthenol available from Alps Pharmaceutical Inc.
*15 Glycerin: Glycerin USP available from Asahi Denka
*16 Butylene Glycol: 1,3-Butylene Glycol available from Kyowa Hakko Kogyo

Preparation Method

The make-up compositions of Examples 6-1-10-1 are prepared as follows:
1) Mixing components numbers 11 through 23 with a suitable mixer and heating to dissolve at 75° C. to make a water phase.
2) Mixing components numbers 7 through 10 with a suitable mixer until homogeneous to make a pigment mixture which is then pulverized using a pulverizer. And then adding the pigment mixture into the water phase with a suitable mixer until homogeneous.
3) Mixing components number 1 through 6 with a suitable mixer and heating to dissolve at 80° C. to make an oil phase which is then added into the water phase and pigment mixture to make an emulsion using a homogenizer.
4) Finally, filling the emulsion into an air-tight container and allowing cooling to room temperature using a cooling unit.

### 3) Examples 11-15

### Oil Dispersion Layer

The following oil dispersion make-up compositions are formed by the process described herein:

| No. | Components | Ex. 11-1 | Ex. 12-1 | Ex. 13-1 | Ex. 14-1 | Ex. 15-1 |
|---|---|---|---|---|---|---|
| 1 | Cyclopentasiloxane *1 | 61.25 | 51.25 | 36.05 | 62.75 | 33.35 |
| 2 | PEG-9 | — | — | — | — | 1.00 |
| | Polydimethylsiloxyethyl | | | | | |
| | Dimethicone *2 | | | | | |
| 3 | Dimethicone and | — | — | 10.00 | 25.00 | — |
| | Dimethicone/Vinyl | | | | | |
| | Dimethicone Crosspolymer *3 | | | | | |
| 4 | Trimethylsiloxysilicate and | — | — | 10.00 | — | 20.00 |
| | Cyclopentasiloxane *4 | | | | | |
| 5 | Phenyl Trimethicone *5 | 5.00 | 5.00 | 5.00 | 5.00 | — |
| 6 | Isotridecyl Isononanoate *6 | 2.00 | 2.00 | 2.00 | 2.00 | 10.00 |
| 7 | Sorbitan Monoisostearate *7 | — | 3.00 | 3.00 | 1.00 | 3.00 |
| 8 | Iron oxide and | 2.00 | 2.00 | 2.00 | — | — |
| | Cyclopentasiloxane and | | | | | |
| | Dimethicone and Disodium | | | | | |
| | Hydrogenated Glutamate *8 | | | | | |
| 9 | Titanium Dioxide and Talc | 10.00 | 10.00 | 12.00 | — | — |
| | and Methicone *9 | | | | | |
| 10 | Mica and Methicone *10 | 8.00 | 8.00 | 8.00 | — | 15.00 |
| 11 | Talc and Methicone *11 | 8.00 | 15.00 | 8.00 | — | 15.00 |
| 12 | Preservative | 0.25 | 0.25 | 0.25 | 0.25 | 0.25 |
| 13 | Candelilla Wax *12 | 1.80 | 1.80 | 1.80 | 2.00 | 1.20 |
| 14 | Ceresin *13 | 1.70 | 1.70 | 1.70 | 2.00 | 1.20 |

| | | | | | |
|---|---|---|---|---|---|
| Total | 100.00 | 100.00 | 100.00 | 100.00 | 100.00 |

Definitions of Components
*1 Cyclopentasiloxane: SH245 available from Dow Corning
*2 PEG-9 Polydimethylsiloxyethyl Dimethicone: KF-6028 available from Shin-Etsu Chemical Co., Ltd.
*3 Dimethicone and Dimethicone/Vinyl Dimethicone Crosspolymer: KSG-16 available from Shin-Etsu Chemical Co., Ltd.
*4 Trimethylsiloxysilicate and Cyclopentasiloxane: Trimethilsiloxysilicate/Cyclomethicone D5 Blend available from GE Toshiba Silicones
*5 Phenyl Trimethicone: KF-56 available from Shin-Etsu Chemical Co., Ltd.
*6 Isotridecyl isononanoate: Crodamol TN available from Croda
*7 Sorbitan monoisostearate: Crill 6 available from Croda
*8 Iron Oxide and Cyclopentasiloxane and Dimethicone and Disodium Hydrogenated Glutamate: SA/NAI-Y-10/D5 (70%), SA/NAI-R-10/D5 (65%) and SA/NAI-B-10/D5 (75%) available from Miyoshi Kasei
*9 Titanium Dioxide and Talc and Methicone: SI-T-CR-50Z available from Miyoshi Kasei
*10 Mica and Methicone: SI Mica available from Miyoshi Kasei
*11 Talc and Methicone: SI-Talc JA13R available from Miyoshi Kasei
*12 Candelilla wax: Candelilla wax NC-1630 available from Cerarica Noda
*13 Ceresin: Ozokerite wax SP-1021 available from Strahl & Pitsh

Preparation Method

The make-up compositions of Examples 11-1-15-1 are prepared as follows:
1) Mixing components numbers 1 through 7 with a suitable mixer until homogeneous to make a silicone phase.
2) Mixing components numbers 8 through 11 with a suitable mixer until homogeneous to make a pigment mixture which is then pulverized using a pulverizer. And then adding the pigment mixture into the silicone phase with a suitable mixer until homogeneous.
3) Adding components number 12 through 14 into the emulsion of step 2 and then heating the emulsion to dissolve the components at 85° C. in a sealed tank.
4) Finally, filling the emulsion in an air-tight container and allowing cooling to room temperature using a cooling unit.

### 4) Examples 1-15

### Pressed Powder Foundation Layer

The following powder foundation make-up compositions are formed by the process described herein.

| | | Ex. 1-2 | Ex. 2-2 | Ex. 3-2 | Ex. 4-2 | Ex. 5-2 |
|---|---|---|---|---|---|---|
| | | Ex. 6-2 | Ex. 7-2 | Ex. 8-2 | Ex. 9-2 | Ex. 10-2 |
| No. | Component | Ex. 11-2 | Ex. 12-2 | Ex. 13-2 | Ex. 14-2 | Ex. 15-2 |
| 1 | Methyl Methacryate | — | — | — | 10.00 | 10.00 |
| | Crosspolymer and | | | | | |
| | Methicone *1 | | | | | |
| 2 | Nylon-12 5 μm *2 | — | — | — | — | 3.00 |
| 3 | Talc and Methicone *3 | 50.09 | 55.60 | 73.60 | 32.10 | 14.59 |
| 4 | Mica and Methicone *4 | 10.00 | — | — | — | 10.00 |
| 5 | Spherical Silica and | 5.00 | — | 5.00 | 2.00 | 5.00 |
| | Methicone *5 | | | | | |
| 6 | Vinyl Dimethicone/ | — | 5.00 | — | — | — |
| | Methicone | | | | | |
| | Silsesquioxane | | | | | |
| | Crosspolymer *6 | | | | | |
| 7 | Mica and Zinc Oxide | — | 20.00 | 8.00 | — | 10.00 |
| | and Methicone and | | | | | |
| | Hydroxyapatite *7 | | | | | |
| 8 | Sericite and Methicone *8 | 5.00 | 7.00 | 5.00 | 32.00 | 20.00 |
| 9 | Titanium Dioxide and | — | — | — | 12.00 | 10.50 |
| | Methicone *9 | | | | | |
| 10 | Mica and Titanium | 15.00 | — | — | — | — |
| | Dioxide *10 | | | | | |
| 11 | Methylparaben | 0.30 | 0.30 | 0.30 | 0.30 | 0.30 |
| 12 | Propylparaben | 0.10 | 0.10 | 0.10 | 0.10 | 0.10 |
| 13 | Iron Oxide and | — | — | — | 2.50 | 2.00 |
| | Methicone *11 | | | | | |
| 14 | Methylphenyl | 8.50 | — | 4.00 | — | 8.50 |
| | polysiloxane *12 | | | | | |
| 15 | Dimethicone *13 | — | 8.00 | — | 5.00 | — |
| 16 | Sorbitan | 1.00 | — | — | — | 1.00 |
| | monoisostearate *14 | | | | | |
| 17 | D-delta-tocopherol *15 | 0.01 | — | — | — | 0.01 |
| 18 | Ethylhexyl | 5.00 | 4.00 | 4.00 | 4.00 | 5.00 |
| | Methoxycinnamate *16 | | | | | |

| | | | | | |
|---|---|---|---|---|---|
| Total | 100.00 | 100.00 | 100.00 | 100.00 | 100.00 |

Definitions of Components
*1 Methyl Methacryate Crosspolymer and Methicone: SI-L-XC-F006Z available from Miyoshi Kasei
*2 Nylon-12 5 μm: NYLON POWDER SP-500 available from TORAY
*3 Talc and Methicone: SI TALC JA13R available from MIYOSHI KASEI, INC.
*4 Mica and Methicone: SI Mica available from MIYOSHI KASEI, INC.
*5 Spherical Silica and Methicone: SI-SILDEX H-52 available from Asahi Glass Company Co., Ltd. and surface treated by Miyoshi Kasei, having an oil absorbency of more than 200 ml/100 g
*6 Vinyl Dimethicone/Methicone Silsesquioxane Crosspolymer: KSP-100 available from Shin-Etsu Chemical Co., Ltd.
*7 Mica and Zinc Oxide and Methicone and Hydroxyapatite: SI-PLV-20 available from MIYOSHI KASEI, INC.
*8 Sericite and Methicone: SI SERICITE available from MIYOSHI KASEI, INC.
*9 Titanium Dioxide and Methicone: SI Titanium Dioxide IS available from MIYOSHI KASEI, INC.
*10 Mica and Titanium Dioxide: FLAMENCO SUPER PEARL available from THE MEARL
*11 Iron Oxide and Methicone: IRON OXIDE series available from DAITO KASEI KOUGYOU CO., LTD.
*12 Methylphenyl polysiloxane: KF56 available from SHINETSU CHEMICAL CO., LTD.
*13 Dimethicone: SH200 available from Dow Corning
*14 Sorbitan Monoisostearate: Crill 6 available from Croda
*15 D-delta-tocopherol: D-DELTA-TOCOPHEROL available from EISAI CO., LTD.
*16 Ethylhexyl Methoxycinnamate: PARSOL MCX available from ROCHE VITAMINS JAPAN K.K.

Preparation Method

The powder make-up compositions of the second layer of Examples 1-15 are prepared as follows:
1) Mixing components numbers 1-13 with a mixer to make a powder component;
2) Separately, mixing components numbers 14-18 under heating until all components are well dissolved to make a binder component;
3) Adding the binder component into the powder component and mixing by a mixer; and
4) Pulverizing the obtained composition in step 3 and filling in a container.

Fifteen different dual-layer foundation products can be made using the preparation method described above and by combining, at the weight ratio of 9:1 to 1:9 of the first layer compositions 1-1 to 15-1 and the corresponding second layer compositions of 1-2 to 15-2 of Examples 1-15. The dual-layer foundation products of the present invention not only have a more attractive aesthetic look, but also provide a variety of skin benefits. For example, Examples 1, 6 and 11 can provide radiant look by comprising mica and titanium dioxide in the second layer, Examples 2, 7 and 12 can provide oily shine control benefit and soft focus benefit by comprising mica and zinc oxide and methicone and hydroxyapatite and spherical silica and methicone and vinyl dimethicone methicone silsesquioxane crosspolymer in the second layer, Examples 3, 8 and 13 can provide oily shine control benefit by comprising mica and zinc oxide and methicone and hydroxyapatite and spherical silica and methicone in the second layer, Examples 4, 9 and 14 can provide soft focus benefit by comprising methyl methacryate crosspolymer and methicone in the second layer, Examples 5, 10 and 15 can provide oily shine control benefit and soft focus benefit by comprising mica and zinc oxide and methicone and hydroxyapatite and spherical silica and methicone and methyl methacryate crosspolymer and methicone in the second layer.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A solid skin care composition comprising:
(a) a first layer which is solid at 45° C. and which is selected from the group consisting of a water-in-oil emulsion, an oil-in-water emulsion and an oil dispersion; and
(b) a second layer which is pressed powder;
wherein the first layer and the second layer are provided in the same package in a manner such that the first layer and the second layer can be simultaneously applied.

2. The composition of claim 1, wherein the first layer and the second layer are visibly distinct.

3. The composition of claim 2, wherein at least one of the first layer and the second layer comprises a colorant to make the layers visibly distinct.

4. The composition of claim 1, wherein the weight ratio of the first layer to the second layer is from about 1:99 to about 99:1.

5. The composition of claim 1, wherein the water-in-oil emulsion, oil-in-water emulsion and oil dispersion each provides a viscosity of from about 100 mPas to about 3000 mPas when brought to a temperature of between about 55° C. and about 90° C.

6. The composition of claim 1, wherein the pressed powder layer comprises:
(a) a powder; and
(b) a non-volatile oil.

7. The composition of claim 6, wherein the first layer is a water-in-oil emulsion comprising:
(a) a volatile silicone oil;
(b) a non-volatile oil;
(c) a solid wax;
(d) a lipophilic surfactant;
(e) a pigment powder; and
(f) water.

8. The composition of claim 6, wherein the first layer is an oil-in-water emulsion comprising:
(a) water;
(b) a hydrophilic surfactant;
(c) a pigment powder;
(d) a non-volatile oil
(e) a fatty compound or fatty acid slats; and
(f) a volatile silicone oil.

9. The composition of claim 6, wherein the composition is an oil dispersion comprising:
(a) a volatile silicone oil;
(b) a non-volatile oil;
(c) a solid wax; and
(d) a powder.

10. The composition of claim 1, wherein at least one of the first layer and the second layer comprises at least one of a benefit agent selected from the group consisting of radiant powder, soft focus powder, soft focus silicone elastomer, film forming polymer, oil absorbing powder, sebum solidifying powder, skin active agent and mixtures thereof.

11. The composition of claim 10, wherein the first layer comprises at least one of a benefit agent selected from the group consisting of film forming polymer, soft focus silicone elastomer, skin active agent and mixtures thereof.

12. The composition of claim 10, wherein the second layer comprises at least one of a benefit agent selected from the group consisting of radiant powder, soft focus powder, oil absorbing powder, sebum solidifying powder and mixtures thereof.
